(19) United States
(12) Patent Application Publication
(10) Pub. No.: US 2011/0071314 A1
(43) Pub. Date: Mar. 24, 2011

(54) PROCESS FOR PRODUCING AROMATIC POLYCARBOXYLIC ACID

(76) Inventors: Akihiro Shibamoto, Hyogo (JP); Takahiro Iwahama, Hyogo (JP)

(21) Appl. No.: 12/994,159

(22) PCT Filed: Jun. 10, 2009
(86) PCT No.: PCT/JP2009/060629

§ 371 Date: Nov. 23, 2010

(30) Foreign Application Priority Data
Jun. 11, 2008 (JP) 2008-153136

(51) Int. Cl. C07C 51/265

(52) U.S. Cl. 562/417

(57) ABSTRACT

A process for producing an aromatic polycarboxylic acid in which all alkyl groups are converted into carboxyl groups in a high yield by decreasing a residual amount of an intermediate product is provided. The process comprises oxygen-oxidizing an aromatic compound having a plurality of alkyl groups (e.g., durene) in the presence of a catalyst containing a cyclic imino unit having an N—OR group (wherein R represents a hydrogen atom or a protecting group for a hydroxyl group) and a transition metal co-catalyst (e.g., a cobalt compound, a manganese compound, and a zirconium compound) under heating in a lower-temperature zone and a higher-temperature zone to produce an aromatic polycarboxylic acid in which a plurality of alkyl groups are oxidized into carboxyl groups. In an initial stage of the reaction, the reaction may be conducted in a first lower-temperature zone (a reaction temperature of 60 to 120° C. and a second lower-temperature zone (an intermediate temperature zone) (a reaction temperature of 100 to 140° C.); and then, in a latter stage of the reaction, the reaction may be conducted in a higher-temperature zone (a reaction temperature of 110 to 150° C.).

### TECHNICAL FIELD

The present invention relates to a process for producing an aromatic polycarboxylic acid by catalytically oxidizing an aromatic compound having a plurality of alkyl groups (e.g., an arene compound) with molecular oxygen.

### BACKGROUND ART

As a process for producing an aromatic polycarboxylic acid by catalytically oxidizing an alkylbenzene with molecular oxygen, various processes are known. For example, U.S. Pat. No. 5,041,633 specification (Patent Document 1) discloses a method for producing an aromatic polycarboxylic acid comprising heating an aromatic compound having at least 3 oxidizable substituents (e.g., durene) in a solvent comprising a C₂₋₆monocarboxylic acid and water in the presence of an oxidation catalyst comprising cobalt, manganese, and a bromine component (e.g., HBr, NaBr), wherein the reaction temperature is initially in the range of from 93 to 199° C. and then water is added, and the reaction temperature is increased at least 14° C. to the range of from 176 to 249° C. This document also discloses an example using zirconium as a catalyst. Further, this document discloses that the method affords a reduction both in the deactivation of the catalyst and in the precipitation of the metal component of the catalyst. However, since this method requires use of a bromine component as a catalyst and addition of an excess of water, it is necessary to use a corrosion-resistant material for a reaction apparatus to be used for the method.

Japanese Patent Application Laid-Open No. 308805/2002 (JP-2002-308805A, Patent Document 2) discloses that an aromatic compound having a plurality of alkyl groups is oxygen-oxidized (or aerobically oxidized) with a cyclic imide compound having an N-hydroxy skeleton as a catalyst in the presence of an acid anhydride to produce a corresponding aromatic polycarboxylic acid or aromatic polycarboxylic anhydride. This document also discloses that pyromellitic acid or an acid anhydride thereof is obtained by oxygen-oxidizing durene using the catalyst and cobalt and manganese as a co-catalyst in acetic anhydride, and that use of an acid anhydride prevents the deactivation of the catalyst (the deactivation of the catalyst due to formation of a complex salt of the metal co-catalyst with the aromatic polycarboxylic acid). Japanese Patent Application Laid-Open No. 273793/2006 (JP-2006-273793A, Patent Document 3) discloses a process for oxygen-oxidizing a substrate using a cyclic acylurea compound having an N-hydroxy skeleton as a catalyst with eliminating water in a reaction system. This document also discloses that an aromatic polycarboxylic acid is obtained from an aromatic compound having 3 or more alkyl groups with a high yield by adding a dehydrating agent (e.g., acetic anhydride). However, this process requires not less than stoichiometric quantities of the dehydrating agent.

Therefore, a process for simply synthesizing an aromatic polycarboxylic acid by a catalytic oxygen-oxidation reaction without addition of a halogen and an acid anhydride (dehydrating agent) in a single step (single-stage synthesis) is required.

WO 2002/040154 (Patent Document 4) discloses that successive addition of a cyclic imide compound as a catalyst to a reaction system often affords an object compound with a higher conversion or selective production (or selectivity), and that a mixture of a cyclic imide compound as a catalyst, cobalt and manganese as co-catalysts, durene, and acetic acid is pressurized at 4 MPa (gauge pressure) in a mixed gas of oxygen and nitrogen, and stirred at 100° C. for one hour and then stirred at 150° C. for 3 hours to give pyromellitic acid in about 53% yield and methyltricarboxybenzene in 26% yield. However, in this process, methyltricarboxybenzene, which is an intermediate product of the oxidation reaction, remains in large quantity, and it is difficult to obtain pyromellitic acid as an object compound in a high yield.

### RELATED ART DOCUMENTS

### Patent Documents

[Patent Document 1] U.S. Pat. No. 5,041,633 specification (Claims and the column “Objects of The Invention”)

[Patent Document 2] JP-2002-308805A (Claims and Examples)

[Patent Document 3] JP-2006-273793A (Claims and [0128])

[Patent Document 4] WO 2002/040154 (page 22, lines 36 to 38 and Example 10)

### SUMMARY OF THE INVENTION

### Problems to be Solved by the Invention

It is therefore an object of the present invention to provide a process for producing an aromatic polycarboxylic acid in a high yield with decreasing a residual amount of an intermediate product (an aromatic carboxylic acid having an alkyl substituent).

Another object of the present invention is to provide a process for producing an aromatic polycarboxylic acid simply and efficiently in a single reaction step (single-stage synthesis).

It is still another object of the present invention to provide a process for producing an aromatic polycarboxylic acid efficiently by a catalytic oxygen-oxidation reaction without addition of a halogen and an acid anhydride (a dehydrating agent).

It is a further object of the present invention to provide a method for increasing selective production of an aromatic polycarboxylic acid.

### Means to Solve the Problems

The inventors of the present invention made intensive studies to achieve the above objects and finally found that, when in the process of the Patent Document 4 [the process for oxygen-oxidizing an aromatic compound having a plurality of alkyl groups (a substrate), e.g., durene, in the presence of a catalyst having a cyclic imino unit and a transition metal co-catalyst] the reaction is conducted at a temperature lower than the conventional reaction temperature, a salt formation of the resulting final oxidation product (e.g., pyromellitic acid) with the transition metal co-catalyst can be prevented without addition of a halogen or a dehydrating agent; that, when the reaction is conducted with feeding a catalyst and oxygen continuously at a lower temperature to reach a predetermined production ratio of the resulting final oxidation product and then at a higher temperature , the final oxidation product is obtained in a high yield in the single-stage reaction step; and that a specified metal co-catalyst system is useful for such a reaction system. The present invention was accomplished based on the above findings.

That is, the present invention includes a process for producing an aromatic polycarboxylic acid, which comprises oxygen-oxidizing an aromatic compound having a plurality of alkyl groups (or oxidizing an aromatic compound having a plurality of alkyl groups with oxygen) in the presence of a transition metal co-catalyst under heating in a plurality of temperature zones with feeding a catalyst and oxygen continuously, wherein the catalyst (hereinafter, the catalyst may simply be referred to as a catalyst having a cyclic imino unit, an imide compound, or a catalyst) comprises a nitrogen atom-containing cyclic compound containing, as a constituent element of the cyclic ring, a skeleton represented by the following formula (1):

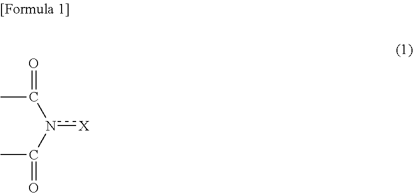

wherein X represents an oxygen atom or an —OR group (wherein R represents a hydrogen atom or a protecting group for a hydroxyl group), and a double line consisting of a solid line and a broken line and connecting “N” and “X” represents a single bond or a double bond.

In this process, assuming that the oxidation degree of the aromatic compound having a plurality of alkyl groups as a substrate is 0% and the oxidation degree of a compound in which all alkyl groups of the aromatic compound are oxidized into carboxyl groups is 100%, the plurality of temperature zones contains at least two temperature zones comprising a first temperature zone for conducting a reaction to reach the oxidation degree of not less than 30% (or a first temperature zone in which the oxidation degree reaches not less than 30%) and a second temperature zone for conducting a reaction to reach the oxidation degree of not less than 75% (or a second temperature zone in which the oxidation degree reaches not less than 75%). That is, according to the process of the present invention, a continuous reaction in which the catalyst and oxygen are fed continuously is conducted in a plurality of temperature zones.

The plurality of temperature zones may contain a lower-temperature zone for conducting a reaction at a reaction temperature within (or selected from) 50 to 140° C. to reach the oxidation degree of 35 to 65%; and a higher-temperature zone for conducting a reaction at a reaction temperature which is higher than the reaction temperature of the lower-temperature zone and is within (or selected from) 100 to 150° C. to reach the oxidation degree of not less than 80%. Moreover, in an initial stage of the reaction (or the oxidation reaction) the plurality of temperature zones may contain at least a first lower-temperature zone for conducting a reaction at a reaction temperature of not higher than 120° C. (or the lower-temperature zone may contain at least a first lower-temperature zone for conducting a reaction at a reaction temperature of not higher than 120° C.). For example, the plurality of temperature zones may contain a first lower-temperature zone for conducting a first reaction (or a first reaction step) at a reaction temperature within 60 to 120° C. (e.g., 60 to 90° C.), a second lower-temperature zone (an intermediate temperature zone) for conducting a second reaction (or a second reaction step) at a reaction temperature within 100 to 140° C. subsequent to the first reaction, and a higher-temperature zone for conducting a third reaction (or a third reaction step) at a reaction temperature within 110 to 150° C. subsequent to the second reaction. The reaction(s) may be carried out under an atmospheric pressure or an applied pressure system. Moreover, in the above-mentioned process, the aromatic compound may be oxygen-oxidized with separately (or independently) feeding the catalyst having a cyclic imino unit and oxygen continuously.

The catalyst having a cyclic imino unit may correspond to a tetracarboxylic anhydride and may be an N-hydroxy cyclic imino compound in which a hydroxyl group may be protected. It is sufficient that the catalyst having a cyclic imino unit is present in the reaction system. The catalyst may be added to the reaction system in a suitable reaction step. For example, the catalyst having a cyclic imino unit may be added to the reaction system at least at a latter stage of the reaction.

The species of the transition metal co-catalyst is not particularly limited to a specific one. The transition metal co-catalyst may comprise a single metal component or a plurality of metal components. For example, the transition metal co-catalyst may comprise a metal component of the Group 9 of the Periodic Table of Elements (e.g., a cobalt compound), a metal component of the Group 7 of the Periodic Table of Elements (e.g., a manganese compound), and a metal component of the Group 4 of the Periodic Table of Elements (e.g., a zirconium compound). In such a combination of the plurality of metal components, the transition metal co-catalyst may comprise a metal component of the Group 9 of the Periodic Table of Elements, a metal component of the Group 7 of the Periodic Table of Elements, and a metal component of the Group 4 of the Periodic Table of Elements, and, for example, the ratio of the metal component of the Group 7 may be 2 to 4 mol relative to 1 mol of the metal component of the Group 9, and the ratio of the metal component of the Group 4 may be 0.5 to 2 mol relative to 1 mol of the total amount of the metal component of the Group 9 and the metal component of the Group 7. In the process of the present invention the reaction may be conducted by adding transition metal co-catalyst to the reaction system at least at a latter stage of the reaction (or in the higher-temperature zone).

The aromatic compound as a substrate may have 2 to 10 alkyl groups on an aromatic ring thereof. Moreover, the catalyst having a cyclic imino unit may have the same number of free carboxyl groups as the alkyl groups of the aromatic compound in the form of a free polycarboxylic acid corresponding to the catalyst. By the reaction, an aromatic polycarboxylic acid, for example, pyromellitic acid can be produced efficiently.

More specifically, the process may comprise oxygen-oxidizing the aromatic compound having methyl groups on ortho position of an aromatic ring thereof in the presence of the transition metal co-catalyst under heating in a pressurized system with feeding a catalyst having the cyclic imino unit and oxygen continuously to produce the aromatic polycarboxylic acid having carboxyl groups on ortho position of an aromatic ring thereof,

wherein the transition metal co-catalyst comprises a cobalt compound, a manganese compound, and a zirconium compound, and the number of moles of the zirconium compound is larger than the total molar quantity of the cobalt compound and the manganese compound, and

assuming that the oxidation degree of the aromatic compound having methyl groups as the substrate is 0% and the oxidation degree of a compound in which all methyl groups of the aromatic compound are oxidized into carboxyl groups is 100%, the reaction (or the oxidation reaction) is conducted in the following temperature zones:

a first lower-temperature zone for conducting a reaction at a reaction temperature within (or selected from) 70 to 90° C.,

a second lower-temperature zone for conducting a reaction at a reaction temperature within (or selected from) 110 to 130° C. to reach the oxidation degree of 35 to 60%, and then

a higher-temperature zone for conducting a reaction at a reaction temperature within (or selected from) 120 to 140° C.

The present invention prevents some unoxidized alkyl groups of the substrate from remaining, and can produce an aromatic polycarboxylic acid in which all alkyl groups of the substrate are converted into carboxyl groups in a high yield. Therefore, the present invention also includes a method for increasing selective production of an aromatic polycarboxylic acid (or a method for increasing selectivity of an aromatic polycarboxylic acid in a production thereof), which comprises oxygen-oxidizing an aromatic compound having a plurality of alkyl groups in the presence of a transition metal co-catalyst under heating in a plurality of temperature zones with feeding a catalyst having the cyclic imino unit and oxygen continuously. In this method, assuming that the oxidation degree of the aromatic compound having a plurality of alkyl groups as a substrate is 0% and the oxidation degree of a compound in which all alkyl groups of the aromatic compound are oxidized into carboxyl groups is 100% , the plurality of temperature zones contains at least two temperature zones comprising a first temperature zone for conducting a reaction to reach the oxidation degree of not less than 30% (or a first temperature zone in which the oxidation degree reaches not less than 30%) and a second temperature zone for conducting a reaction to reach the oxidation degree of not less than 75% (or a second temperature zone in which the oxidation degree reaches not less than 75%).

Incidentally, hereinafter the term “aromatic polycarboxylic acid” means not only a polycarboxylic acid having a free carboxyl group but also a compound having a free carboxyl group and an acid anhydride group and an aromatic acid anhydride having an acid anhydride group.

### Effects of the Invention

The present invention affords a high yield of a aromatic polycarboxylic acid since a reaction in a plurality of reaction temperature zones prevents a production of a polyvalent metal salt of the aromatic polycarboxylic acid (an insoluble matter or aprecipitate) and reduces a residual amount of an intermediate product (an aromatic carboxylic acid having an alkyl substituent). Moreover, the present invention produces an aromatic polycarboxylic acid simply and efficiently in a single reaction step (single synthesis or one pot) without eliminating a precipitate and others. Further, a catalytic oxygen-oxidation reaction efficiently produces an aromatic polycarboxylic acid without addition of a halogen and an acid anhydride (a dehydrating agent). Furthermore, the selective production of the aromatic polycarboxylic acid is increased due to an inhibited production of an intermediate product or a by-product.

### DESCRIPTION OF EMBODIMENTS

According to the present invention, an aromatic polycarboxylic acid is produced by catalytically oxygen-oxidizing an aromatic compound having a plurality of alkyl groups in the presence of a catalyst having a cyclic imino unit (an imide compound) and a transition metal co-catalyst.

[Catalyst having Cyclic Imino Unit]

The imide compound has a cyclic imino unit containing a skeleton represented by the above-mentioned formula (1) (skeleton (1)) as a constituent element of a ring thereof. It is sufficient that the imide compound has at least one skeleton (1) in a molecule thereof, and the imide compound may have a plurality of skeletons (1). Moreover, the cyclic imino unit may form one ring by a plurality of skeletons (1) as a constituent element. The cyclic imino unit may have one or a plurality of hetero atom(s) (for example, a nitrogen atom, a sulfur atom, and an oxygen atom (particularly, a nitrogen atom)) other than a nitrogen atom of the skeleton (1), as a constituent atom of a ring thereof.

In the skeleton (1) [or the cyclic imino unit of the catalyst (imide compound) ], X represents an oxygen atom, an —OH group, or a hydroxyl group protected by a protecting group R. The protecting group R may be referred to, for example, the above-mentioned Patent Document 2, Patent Document 3, and Patent Document 4. The protecting group R may include, for example, a hydrocarbon group which may have a substituent [for example, an alkyl group, an alkenyl group (e.g., allyl group), a cycloalkyl group, an aryl group which may have a substituent, and an aralkyl group which may have a substituent]; a group which can form an acetal or hemiacetal group with a hydroxyl group, for example, a C₁₋₃alkyl group having a substituent [e.g., a halo C₁₋₂alkyl group (e.g., 2,2,2-trichloroethyl group), a C₁₋₄alkoxyC₁₋₂alkyl group (e.g., methoxymethyl group, ethoxymethyl group, isopropoxymethyl group, 2-methoxyethyl group, 1-ethoxyethyl group, and 1-isopropoxyethyl group), a C₁₋₄alkylthioC₁₋₂alkyl group corresponding to such a C₁₋₄alkoxyC₁₋₂alkyl group, a haloC₁₋₄alkoxyC₁₋₂alkyl group (e.g., 2,2,2-trichloroethoxymethyl group, and bis(2-chloroethoxy)methyl group), a C₁₋₄alkylC₁₋₄alkoxyC₁₋₂alkyl group (e.g., 1-methyl-1-methoxyethyl group), a C₁₋₄alkoxyC₁₋₃alkoxyC₁₋₂alkyl group (e.g., 2-methoxyethoxymethyl group), a C₁₋₄alkylsilylC₁₋₄alkoxyC₁₋₂alkyl group (e.g., 2-(trimethylsilyl)ethoxymethyl group), and an aralkyloxyC₁₋₂alkyl group (e.g., benzyloxymethyl group) ], a 5- or 6-membered heterocycle group having a hetero atom selected from the group consisting of a nitrogen atom, an oxygen atom and a sulfur atom (e.g., a saturated heterocycle group such as tetrahydropyranyl group or tetrahydrofuranyl group), and a 1-hydroxy-C₁₋₂₀alkyl group which may have a substituent (e.g., a 1-hydroxy-C₁₋₁₀alkyl group such as 1-hydroxyethyl or 1-hydroxyhexyl group, and 1-hydroxy-1-phenylmethyl group); an acyl group (for example, a saturated or unsaturated alkylcarbonyl group, e.g., a C₁₋₂₀alkyl-carbonyl group such as formyl, acetyl, propionyl, butyryl, or isobutyryl group; acetoacetyl group; an alicyclic acyl group, e.g., a C₄₋₁₀cycloalkyl-carbonyl group such as cyclopentanecarbonyl or cyclohexanecarbonyl group; and a C₆₋₁₂aryl-carbonyl group such as benzoyl or naphthoyl group), a sulfonyl group having an alkyl group which may be halogenated (e.g., an alkylsulfonyl group such as methanesulfonyl group or trifluoromethanesulfonyl group, and an arylsulfonyl group such as benzenesulfonyl, p-toluenesulfonyl, or naphthalenesulfonyl group); an alkoxycarbonyl group (e.g., a C₁₋₄alkoxy-carbonyl group such as methoxycarbonyl group or ethoxycarbonyl group), an aralkyloxycarbonyl group (e.g., benzyloxycarbonyl group, and p-methoxybenzyloxycarbonyl group); a carbamoyl group which has either a substituent or no substituent (or a substituted or non-substituted carbamoyl group) (e.g., carbamoyl group, a C₁₋₄alkylcarbamoyl group such as methylcarbamoyl group, and phenylcarbamoyl group); a residual group obtained by eliminating a hydroxyl group from an inorganic acid (e.g., sulfuric acid, nitric acid, phosphoric acid, and boric acid); a dialkylphosphanothioyl group, a diarylphosphanothioyl group; and a silyl group having a substituent (or a substituted silyl group) (e.g., trimethylsilyl, t-butyldimethylsilyl, tribenzylsilyl, and triphenylsilyl group).

The preferred R may include a protecting group other than an alkyl group (e.g., methyl group), for example, a hydrogen atom; a group capable of forming an acetal or hemiacetal group with a hydroxyl group; and a hydrolyzable protecting group, which can be eliminated by hydrolysis, for example, a residual group obtained by eliminating a hydroxyl group from an acid such as a carboxylic acid, a sulfonic acid, a carbonic acid, a carbamic acid, a sulfuric acid, a phosphoric acid, or a boric acid (e.g., an acyl group, a sulfonyl group, an alkoxycarbonyl group, and a carbamoyl group).

In the formula, the double line consisting of a solid line and a broken line and connecting the nitrogen atom “N” and “X” represents a single bond or a double bond.

The catalyst (imide compound) having the cyclic imino unit may include, for example, a compound having 5-membered or 6-membered cyclic unit containing the skeleton(1) as a constituent element of the ring thereof. Such a compound is known and may be referred to the above-mentioned Patent Documents 2, 3 and 4, and others. The compound having the 5-membered cyclic unit may include, for example, a compound represented by the following formula (2). The compound having the 6-membered cyclic unit may include, for example, a compound represented by the following formula (3) or (4).

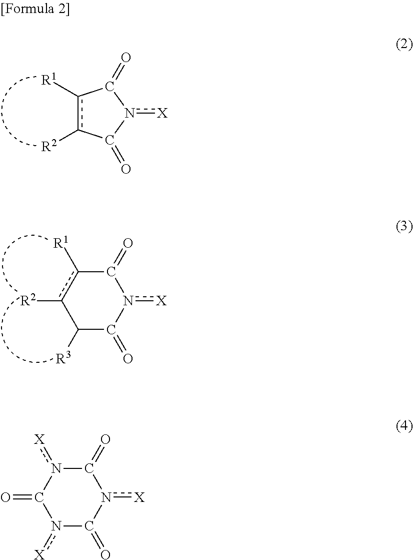

In the formulae, R¹, R² and R³ are the same or different and each represent a hydrogen atom, a halogen atom, an alkyl group, an aryl group, a cycloalkyl group, a hydroxyl group, an alkoxy group, a carboxyl group, an oxycarbonyl group having a substituent (or a substituted oxycarbonyl group), an acyl group, or an acyloxy group, R¹ and R² may bond together to form an aromatic or non-aromatic ring with the adjacent carbon atoms, R² and R³ may bond together to form an aromatic or non-aromatic ring with the adjacent carbon atoms. These rings may further have one or two cyclic imino units mentioned above. The double line consisting of a solid line and a broken line represents a single bond or a double bond. The group X has the same meaning as defined above.

The halogen atom represented by each of the substituents R¹, R² and R³ may include an iodine atom, a bromine atom, a chlorine atom, and a fluorine atom. The alkyl group may include, for example, a straight chain or branched chain C₁₋₂₀alkyl group such as methyl, ethyl, propyl, isopropyl, butyl, isobutyl, s-butyl, t-butyl, hexyl, or decyl group (particularly, a C₁₋₁₆alkyl group). The cycloalkyl group may include a C₃₋₁₀ cycloalkyl group such as cyclopentyl or cyclohexyl group. The aryl group may include phenyl group, naphthyl group, and others.

The alkoxy group may include, for example, a straight chain or branched chain C₁₋₂₀alkoxy group such as methoxy, ethoxy, isopropoxy, butoxy, t-butoxy, hexyloxy, octyloxy, decyloxy, dodecyloxy, tetradecyloxy, or octadecyloxy group (particularly, a C₁₋₁₆alkoxy group). The substituted oxycarbonyl group may include, for example, a C₁₋₂₀alkoxy-carbonyl group such as methoxycarbonyl, ethoxycarbonyl, isopropoxycarbonyl, butoxycarbonyl, t-butoxycarbonyl, hexyloxycarbonyl, octyloxycarbonyl, or decyloxycarbonyl group; a C₃₋₁₀cycloalkyloxy-carbonyl group such as cyclopentyloxycarbonyl or cyclohexyloxycarbonyl group; a C₆₋₁₂aryloxy-carbonyl group such as phenyloxycarbonyl or naphthyloxycarbonyl group; and a C₆₋₁₂arylC₁₋₄alkyloxy-carbonyl group such as benzyloxycarbonyl group. The acyl group may include, for example, a C₁₋₂₀alkyl-carbonyl group such as formyl, acetyl, propionyl, butyryl, isobutyryl, valeryl, pivaloyl, hexanoyl, or octanoyl group; acetoacetyl group; a cycloalkylcarbonyl group such as cyclopentylcarbonyl or cyclohexylcarbonyl group (e.g., a C₃₋₁₀cycloalkyl-carbonyl group); and an aromatic acyl group such as benzoyl or naphthoyl group (e.g., an arylcarbonyl group). The acyloxy group may include an acyloxy group corresponding to the acyl group, for example, a C₁₋₂₀alkyl-carbonyloxy group; acetoacetyloxy group; a cycloalkyl-carbonyloxy group; and an arylcarbonyloxy group.

The substituents R¹, R² and R³ may be the same or different from one another. Moreover, in the above-mentioned formulae (2) to (4), a broken line connecting R¹ with R² represents that R¹ and R² may bond together to form an aromatic or non-aromatic ring with the adjacent carbon atoms, and a broken line connecting R² with R³ represents that R² and R³ may bond together to form an aromatic or non-aromatic ring with the adjacent carbon atoms. Incidentally, the ring formed by bonding of R¹ and R² and the ring formed by bonding of R² and R³ may be combined to form a polycyclic aromatic or non-aromatic condensed ring.

Each of the aromatic or non-aromatic ring formed by bonding of R¹ and R² and the aromatic or non-aromatic ring formed by bonding R² and R³ may be, for example, an about 5- to 16-membered ring, preferably an about 6- to 14-membered ring, and more preferably an about 6- to 12-membered ring (e.g., an about 6- to 10-membered ring). Moreover, the aromatic or non-aromatic ring may be a heterocycle or a condensed heterocycle, and is practically a hydrocarbon ring or a hydrocarbon ring further having one or two cyclic imino units. Such a hydrocarbon ring may include, for example, a non-aromatic alicyclic ring (e.g., a C₃₋₁₀cycloalkane ring such as cyclohexane ring, a C₃₋₁₀cycloalkene ring such as cyclohexene ring); a non-aromatic bridged ring (e.g., a bicyclic to tetracyclic bridged hydrocarbon ring such as 5-norbornene ring), and an aromatic ring (e.g., a C₆₋₁₂arene ring such as benzene ring or naphthalene ring, and a condensed ring). These rings may have a substituent (e.g., an alkyl group, a haloalkyl group, a hydroxyl group, an alkoxy group, a carboxyl group, a substituted oxycarbonyl group, an acyl group, an acyloxy group, a nitro group, a cyano group, an amino group, and a halogen atom). The ring comprises an aromatic ring in practical cases.

The preferred catalyst (imide compound) may include the compounds represented by the following formulae (1a) to (1d) and the compound represented by the above formula (4):

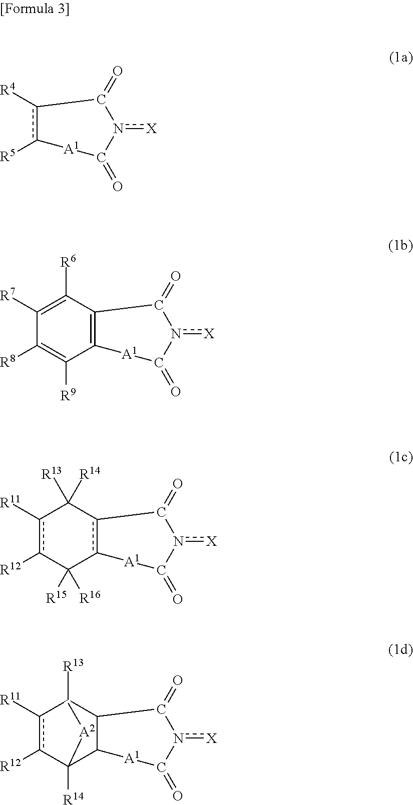

wherein —A¹— represents a single bond or a group represented by the following formula (A):

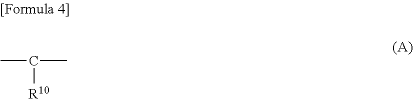

R⁴ to R¹⁶ are the same or different and each represent a hydrogen atom, an alkyl group as exemplified above, a haloalkyl group, a hydroxyl group, an alkoxy group as exemplified above, a carboxyl group, a substituted oxycarbonyl group as exemplified above, an acyl group as exemplified above, an acyloxy group as exemplified above, a nitro group, a cyano group, an amino group, and a halogen atom as exemplified above; two vicinal groups of R⁶ to R¹² (or two groups selected from R⁶ to R¹² and attached to adjacent carbon atoms) may bond together to form the same aromatic or non-aromatic ring as described in the above, with the adjacent carbon atoms, or may form a cyclic imino unit represented by the following formula (1e):

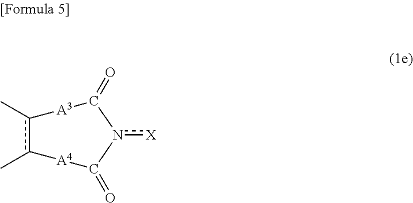

(wherein, —A³— and —A⁴— each represent a single bond or a group represented by the above-mentioned formula (A); provided that when —A³— represents a single bond, —A⁴— represents a single bond or a group represented by the formula (A), and when —A³— represents a group represented by the formula (A), —A⁴— represents a single bond.); the aromatic or non-aromatic ring formed by bonding of two vicinal groups of R⁶ to R¹² may further has one or two cyclic imino units represented by the formula (1e); in the formula (1d), A² represents a methylene group or an oxygen atom; and a double line consisting of a solid line and a broken line represents a single bond or a double bond.

Incidentally, the imide compound having a plurality of cyclic imino units may include, for example, compounds represented by the following formulae:

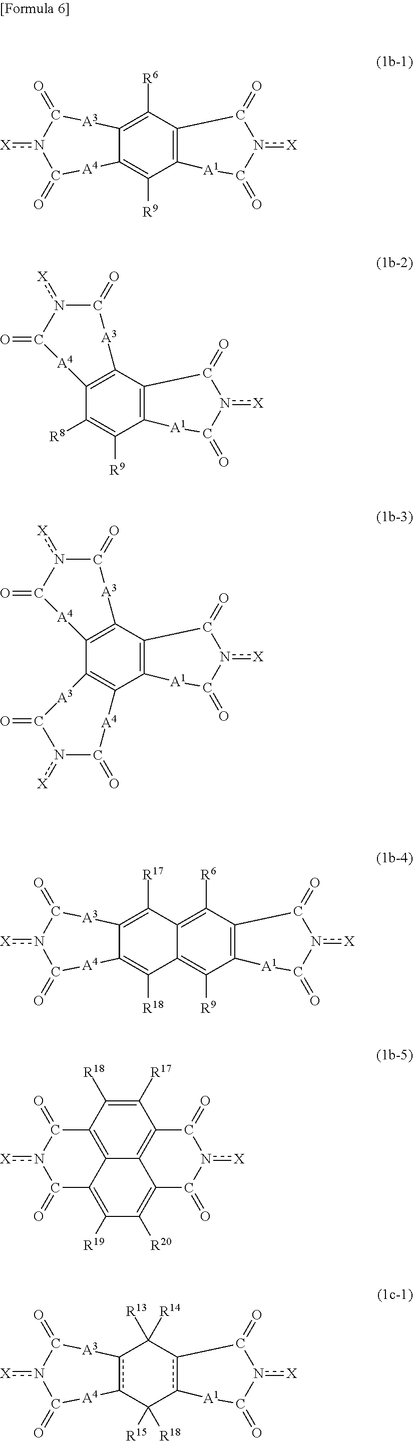

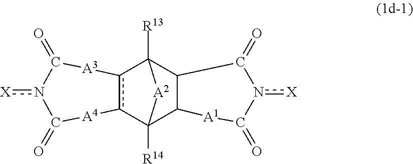

wherein R¹⁷ to R²⁰ are the same or different and each represent a hydrogen atom, an alkyl group as exemplified above, a haloalkyl group, a hydroxyl group, an alkoxy group as exemplified above, a carboxyl group, a substituted oxycarbonyl group as exemplified above, an acyl group as exemplified above, an acyloxy group as exemplified above, a nitro group, a cyano group, an amino group, and a halogen atom as exemplified above; —A¹—, A², —A³—, —A⁴—, R⁶, R⁸, R⁹, R¹³ to R¹⁶ and X have the same meanings as defined above; two vicinal groups of R⁶, R⁷ to R¹⁰, and R¹⁷ to R²⁰ may bond together to form the same aromatic or non-aromatic ring as described in the above, with the adjacent carbon atoms; and a double line consisting of a solid line and a broken line represents a single bond or a double bond.

In the substituents R⁴ to R²⁰, the haloalkyl group may include a haloC₁₋₂₀alkyl group such as trifluoromethyl group. Usually the substituents R⁴ to R²⁰ are independently a hydrogen atom, an alkyl group, a carboxyl group, a substituted oxycarbonyl group, a nitro group, or a halogen atom.

Examples of the preferred imide compound include a compound in which X is OH group in each of the formulae, for example, N-hydroxysuccinimide or a compound having an acyloxy group (such as acetoxy, propionyloxy, valeryloxy, pentanoyloxy, or lauroyloxy group) or an arylcarbonyloxy group (such as benzoyloxy group) as substituents on α,β-positions of N-hydroxysuccinimide, N-hydroxymaleimide, N-hydroxyhexahydrophthalimide, N,N′-dihydroxycyclohexanetetracarboximide, N-hydroxyphthalimide or a compound having an alkoxycarbonyl group (such as methoxycarbonyl, ethoxycarbonyl, pentyloxycarbonyl, or dodecyloxycarbonyl group) or an aryloxycarbonyl group (such as phenoxycarbonyl group) as a substituent on 4-position and/or 5-position of N-hydroxyphthalimide, N-hydroxytetrabromophthalimide, N-hydroxytetrachlorophthalimide, N-hydroxyhetimide (N-hydroxyhet acid imide), N-hydroxyhimimide (N-hydroxyhimic acid imide), N-hydroxytrimellitimide, N,N′-dihydroxypyromellitimide, an N,N′-dihydroxynaphthalenetetracarboximide (e.g., N,N′-dihydroxynaphthalene-1,8,4,5-tetracarboximide), and 1,3,5-trihydroxyisocyanuric acid; a compound in which X is OR group in the formula (1) (wherein R represents an acyl group such as acetyl group), for example, a compound having an N-acyl skeleton, corresponding to the above-exemplified compound having an N-hydroxy skeleton (i.e., a compound in which X is OH group in the formula (1)) (e.g., N-acetoxysuccinimide, N-acetoxymaleimide, N-acetoxyhexahydrophthalimide, N,N′-diacetoxycyclohexanetetracarboximide, N-acetoxyphthalimide, N-acetoxytetrabromophthalimide, N-acetoxytetrachlorophthalimide, N-acetoxyhetimide (N-acetoxyhet acid imide), N-acetoxyhimimide (N-acetoxyhimic acid imide), N-acetoxytrimellitimide, N,N′-diacetoxypyromellitimide, an N,N′-diacetoxynaphthalenetetracarboximide (e.g., N,N′-diacetoxynaphthalene-1,8,4,5-tetracarboximide), N-valeryloxyphthalimide, and N-lauroyloxyphthalimide); a compound corresponding to the above-exemplified compound having an N-hydroxy skeleton (i.e., a compound in which X is OH group in the formula (1)) and being represented by the formula (1) in which X is OR group (wherein R represents a group capable of forming an acetal or hemiacetal group with a hydroxyl group), for example, N-methoxymethyloxyphthalimide, N-(2-methoxyethoxymethyloxy)phthalimide, and N-tetrahydropyranyloxyphthalimide; a compound represented by the formula (1) in which X is OR group (wherein R represents sulfonyl group), for example, N-methanesulfonyloxyphthalimide, and N-(p-toluenesulfonyloxy)phthalimide; a compound represented by the formula (1) in which X is OR group (wherein R represents a residual group obtained by eliminating a hydroxyl group from an inorganic acid), for example, an ester of N-hydroxyphthalimide with sulfuric acid, nitric acid, phosphoric acid, boric acid, and other acids.

The process for producing the catalyst (imide compound) having the cyclic imino unit is described in the above-mentioned Patent Documents 2, 3 and 4 or others, and the catalyst can be produced in accordance with the processes described in these documents. Incidentally, the acid anhydride corresponding to the catalyst may include, for example, a saturated or unsaturated aliphatic dicarboxylic anhydride such as succinic anhydride or maleic anhydride; a saturated or unsaturated non-aromatic cyclic polycarboxylic anhydride (an alicyclic polycarboxylic anhydride) such as tetrahydrophthalic anhydride, hexahydrophthalic anhydride (1,2-cyclohexanedicarboxylic anhydride), 1,2,3,4-cyclohexanetetracarboxylic acid 1,2-anhydride, or methylcyclohexenetricarboxylic anhydride; a bridged cyclic polycarboxylic anhydride (an alicyclic polycarboxylic anhydride) such as het anhydride (het acid anhydride) or himic anhydride (himic acid anhydride); and an aromatic polycarboxylic anhydride such as phthalic anhydride, tetrabromophthalic anhydride, tetrachlorophthalic anhydride, nitrophthalic anhydride, het acid, himic anhydride, trimellitic anhydride, pyromellitic anhydride, mellitic anhydride, 1,8;4,5-naphthalenetetracarboxylic dianhydride, or 2,3;6,7-naphthalenetetracarboxylic dianhydride.

In the present invention, the preferred catalyst (a catalyst having a cyclic imino unit or a compound having an N-hydroxy skeleton) includes an alicyclic or aromatic compound. In particular, such a catalyst includes a compound having a plurality of cyclic imino units, for example, an N-hydroxy cyclic imino compound which corresponds to a tetracarboxylic anhydride and in which a hydroxyl group may be protected (an N-hydroxy cyclic imino compound, or a compound in which a hydroxyl group of an N-hydroxy cyclic imino compound is protected by a protecting group).

Further, the catalyst having a cyclic imino unit (the imide compound) preferably includes a compound derived from an acid anhydride having the same number of free carboxyl groups as the number of alkyl groups of an aromatic compound as a substrate (the number of alkyl substituents). More specifically, it is preferable that the catalyst having a cyclic imino unit (the imide compound) have the same number of free carboxyl groups (or acid anhydride groups) as the number of alkyl groups of an aromatic compound as a substrate (the number of alkyl substituents) in the form of a free polycarboxylic acid (or acid anhydride) corresponding to the catalyst. That is, the catalyst having a cyclic imino unit (the imide compound) is preferably a compound derived from the same kind of acid anhydride corresponding to the imide compound. Further, the catalyst is preferably a compound having at least one cyclic imino unit (or imide ring) and one or a plurality of free carboxyl group(s), particularly, a compound having a plurality of cyclic imino units (or imide rings).

The preferred catalyst (the imide compound) may include a compound derived from trimellitic acid or an acid anhydride thereof (e.g., N-hydroxytrimellitimide and N-acetoxytrimellitimide), a compound derived from cyclohexanetetracarboxylic acid or an acid anhydride thereof (e.g., N,N′-dihydroxycyclohexanetetracarboximide and N,N′-diacetoxycyclohexanetetracarboximide), a compound derivedfrompyromellitic acid or an acid anhydride thereof (e.g., N,N′-dihydroxypyromellitimide and N,N′-diacetoxypyromellitimide), a compound derived from naphthalenetetracarboxylic acid (e.g., 1,8,4,5-tetracarboxynaphthalene) or an acid anhydride thereof (e.g., an N,N′-dihydroxynaphthalenetetracarboximide such as N,N′-dihydroxynaphthalene-1,8,4,5-tetracarboximide, and an N,N′-diacetoxynaphthalenetetracarboximide such as N,N′-diacetoxynaphthalene-1,8,4,5-tetracarboximide)).

Further, the catalyst (imide compound) also includes a cyclic compound having the skeleton represented by the formula (1) through a linking group or linking skeleton (for example, a biphenyl unit and a bisaryl unit). As the catalyst (imide compound), there may be a compound derived from a tetracarboxybiphenyl compound or an acid anhydride thereof, for example, N,N′-dihydroxybiphenyl tetracarboximide and N,N′-diacetoxybiphenyl tetracarboximide, and in addition, a compound derived from a biphenyl ether tetracarboxylic acid or an acid anhydride thereof (e.g., N,N′-dihydroxybiphenyl ether tetracarboximide and N,N′-diacetoxybiphenyl ether tetracarboximide), a compound derived from a biphenyl sulfone tetracarboxylic acid or an acid anhydride thereof (e.g., N,N′-dihydroxybiphenyl sulfone tetracarboximide and N,N′-diacetoxybiphenyl sulfone tetracarboximide), a compound derived from a biphenyl sulfide tetracarboxylic acid or an acid anhydride thereof (e.g., N,N′-dihydroxybiphenyl sulfide tetracarboximide and N,N′-diacetoxybiphenyl sulfide tetracarboximide), a compound derived from a biphenyl ketone tetracarboxylic acid or an acid anhydride thereof (e.g., N,N′-dihydroxybiphenyl ketone tetracarboximide and N,N′-diacetoxybiphenyl ketone tetracarboximide), a compound derived from a bis (3,4-dicarboxyphenyl) alkane or an acid anhydride thereof (e.g., N,N′-dihydroxybiphenylalkanetetracarboximide and N,N′-diacetoxybiphenylalkanetetracarboximide), and others.

The imide compound represented by the formula (1) may be used alone or in combination. The imide compound may be produced in the reaction system.

The imide compound may be used in the form that the compound is supported on a support (or a carrier) (for example, a porous support such as an activated carbon, a zeolite, a silica, a silica-alumina, or a bentonite). The amount to be supported of the imide compound relative to 100 parts by weight of the support is, for example, about 0.1 to 50 parts by weight, preferably about 0.5 to 30 parts by weight, and more preferably about 1 to 20 parts by weight.

The amount of the catalyst (the imide compound) may be selected from a wide range of about 0.01 to 100 mol % relative to the reaction component (the substrate; the aromatic compound) in terms of cyclic imino unit, and, for example, be about 0.2 to 100 mol % (e.g., about 0.5 to 75 mol %), preferably about 1 to 50 mol % (e.g., about 2.5 to 40 mol %), and more preferably about 5 to 30 mol % (e.g., about 7 to 30 mol %) relative to the substrate.

The catalyst may be added in various forms (or modes), such as a bulk feeding or a successive addition, to the reaction system, and usually added by a continuous addition. For the continuous addition, the addition time may be, for example, about 1 to 10 hours and preferably about 2 to 7 hours.

[Transition Metal Co-Catalyst]

The transition metal co-catalyst may also be referred to the above-mentioned Patent Documents 2, 3 and 4, and others. As the transition metal co-catalyst, in practical cases a metal compound having a metal element of the Groups 2 to 15 of the Periodic Table of Elements is used. Incidentally, hereinafter, boron (B) is regarded as a metal element. The metal element may include, for example, an element of the Group 2 (e.g., Mg, Ca, Sr, and Ba), an element of the Group 3 (e.g., Sc, a lanthanoid, and an actinoid), an element of the Group 4 (e.g., Ti, Zr, and Hf), an element of the Group 5 (e.g., V), an element of the Group 6 (e.g., Cr, Mo, and W), an element of the Group 7 (e.g., Mn), an element of the Group 8 (e.g., Fe, Ru, and Os), an element of the Group 9 (e.g., Co, Rh, and Ir), an element of the Group 10 (e.g., Ni, Pd, and Pt), an element of the Group 11 (e.g., Cu), an element of the Group 12 (e.g., Zn), an element of the Group 13 (e.g., B, Al, and In), an element of the Group 14 (e.g., Sn and Pb), and an element of the Group 15 (e.g., Sb and Bi), of the Periodic Table of Elements. The transition metal element (the element of the Groups 3 to 12 of the Periodic Table of Elements), particularly, Mn, Co, Zr, Ce, Fe, V, and Mo (among others, Mn, Co, Zr, Ce, and Fe) are preferred. The valence of the metal element is not particularly limited to a specific one, and may be, for example, about 0 to 6.

The metal compound may include an inorganic compound such as a simple substance (or an elemental substance) of the metal element, a hydroxide of the metal element, an oxide of the metal element (including a compound oxide), a halide of the metal element (a fluoride, a chloride, a bromide, and an iodide), a salt of the metal element with an oxo acid (e.g., a nitrate, a sulfate, a phosphate, a borate, and a carbonate), a salt of the metal element with an isopoly acid, or a salt of the metal element with a heteropoly acid; and an organic compound such as a salt of the metal element with an organic acid (e.g., an acetate, a propionate, a cyanate, a naphthenate, and a stearate) or a complex of the metal element. The ligand of the complex may include OH (hydroxo), an alkoxy (e.g., methoxy, ethoxy, propoxy, and butoxy), an acyl (e.g., acetyl and propionyl), an alkoxycarbonyl (e.g., methoxycarbonyl and ethoxycarbonyl), an acetylacetonato, a cyclopentadienyl group, a halogen atom (e.g., chlorine and bromine), CO, CN, oxygen atom, H₂O (aquo), a phosphorus compound such as a phosphine (e.g., a triarylphosphine such as triphenylphosphine), a nitrogen-containing compound such as NH₃ (ammine), NO, NO₂ (nitro), NO₃ (nitrato), ethylenediamine, diethylenetriamine, pyridine, or phenanthroline, and others.

Typical examples of the metal compound may include an inorganic compound such as a hydroxide [e.g., cobalt hydroxide and vanadium hydroxide], an oxide [e.g., cobalt oxide, vanadiumoxide, manganese oxide, and zirconiumoxide], a halide (e.g., cobalt chloride, cobalt bromide, vanadium chloride, vanadyl chloride, and zirconium chloride), or an salt of an inorganic acid (e.g., cobalt nitrate, cobalt sulfate, cobalt phosphate, vanadium sulfate, vanadyl sulfate, sodium vanadate, manganese sulfate, and zirconium sulfate); a salt of an organic acid [e.g., cobalt acetate, cobalt naphthenate, cobalt stearate, manganese acetate, zirconium acetate, and zirconium hydroxyacetate]; a complex [e.g. , abivalent or tervalent cobalt compound such as cobalt acetylacetonato, a bi- to quinquevalent vanadium compound such as vanadium acetylacetonato or vanadyl acetylacetonato, a bivalent or tervalent manganese compound such as manganese acetylacetonato, and a quadrivalent or quinquevalent zirconium compound such as zirconium acetylacetonato]; and others.

The metal compound may be used alone or in combination. In particular, use of a combination of a metal component of the Group 9 of the Periodic Table of Elements (e.g., a cobalt compound) and a metal component of the Group 7 of the Periodic Table of Elements (e.g., a manganese compound), among others, use of a combination of a metal component of the Group 9 of the Periodic Table of Elements (e.g., a cobalt compound), a metal component of the Group 7 of the Periodic Table of Elements (e.g., a manganese compound) and a metal component of the Group 4 of the Periodic Table of Elements (e.g., a zirconium compound), can improve the catalyst activity to increase the yield of the aromatic polycarboxylic acid. Moreover, a plurality of metal compounds different in valence may be used in combination. For example, a bivalent or tervalent cobalt compound (e.g., cobalt acetate (II)), a bivalent or tervalent manganese compound (e.g., manganese acetate (II)), and a quadrivalent or quinquevalent zirconium compound (e.g., zirconium oxoacetate (IV) or zirconium sulfate (IV)) may be used in combination.

The plurality of metal components (or metal compounds) may be used in suitable quantitative proportions as long as the catalyst activities are not inhibited. When the transition metal co-catalyst comprises a cobalt compound, a manganese compound, and a zirconium compound, for example, the number of moles of the zirconium may be larger than the total molar quantity of the cobalt and the manganese in terms of metal elements. For example, the ratio of the metal component of the Group 7 of the Periodic Table of Elements (the manganese compound) may be about 0.5 to 6 mol (e.g., about 1 to 5 mol, preferably about 2 to 4 mol, and more preferably about 2.5 to 3.5 mol), in terms of metal elements, relative to 1 mol of the metal component of the Group 9 of the Periodic Table of Elements (the cobalt compound). Moreover, the ratio of the metal component of the Group 4 of the Periodic Table of Elements (the zirconium compound) may be about 0.1 to 3 mol (e.g., about 0.3 to 2.5 mol, preferably about 0.5 to 2 mol, and more preferably about 1 to 2 mol) relative to 1 mol of the total amount of the metal component of the Group 9 of the Periodic Table of Elements and the metal component of the Group 7 of the Periodic Table of Elements (the cobalt compound and the manganese compound) in terms of metal elements.

The amount of the transition metal co-catalyst may be, for example, selected from the range of about 0.001 to 10 mol , preferably about 0.005 to 5 mol , and more preferably about 0.01 to 3 mol relative to 1 mol of the imide compound in terms of metal elements. The amount of the transition metal co-catalyst may be about 5 to 1000 ppm and preferably about 10 to 500 ppm (e.g., about 20 to 300 ppm) relative to the imide compound. Moreover, the amount of the metal compound may be, for example, selected from the range of about 1×10⁻⁷ to 0.1 mol (e.g., about 0.001 to 0.05 mol) relative to 1 mol of the reaction component (the substrate) in terms of metal elements. The amount of the transition metal co-catalyst (usually, including use of a plurality of co-catalyst components) is about 0.001 to 20 mol %, preferably about 0.01 to 10 mol % , and more preferably about 0.05 to 5 mol % relative to the substrate in terms of metal elements.

The transition metal co-catalyst may be added in various forms (or modes) to the reaction system, and the forms (or modes) may include, for example, a bulk feeding, a successive addition, and a continuous addition. Incidentally, in some cases, the co-catalyst component and the resulting aromatic polycarboxylic acid form a salt along with the progress of the reaction, so that the co-catalyst component does not act effectively. Therefore, when the catalyst activity is decreased, the transition metal co-catalyst may be added in the form (or mode) such as a successive addition or a continuous addition to the reaction system.

In the present invention, an organic salt may be used as a co-catalyst. The organic salt comprises a polyatomic cation or polyatomic anion containing an element of the Group 15 (e.g., N, P, As, and Sb) or the Group 16 (e.g., S) of the Periodic Table of Elements bonding to at least one organic group, and a counter ion. Representative examples of the organic salt may include an organic onium salt such as an organic ammonium salt, an organic phosphonium salt, or an organic sulfonium salt. The organic salt may also include a salt of an alkylsulfonic acid; a salt of an arylsulfonic acid which may have a C₁₋₂₀alkyl group as a substituent; a sulfonate-based ion exchange resin (an ion exchanger); a phosphonate-based ion exchange resin (an ion exchanger); and others. The amount of the organic salt is, for example, about 0.001 to 10 mol, preferably about 0.005 to 5 mol, and more preferably about 0.005 to 3 mol, relative to 1 mol of the imide compound.

In the present invention, a strong acid may be used as a co-catalyst. The strong acid may include, for example, a hydrogen halide , a hydrohalogenic acid, a sulfuric acid, and a heteropoly acid. The amount of the strong acid is, for example, about 0.001 to 3 mol, preferably about 0.005 to 2.5 mol, and more preferably about 0.01 to 2 mol, relative to 1 mol of the imide compound.

In the present invention, further, a carbonyl compound having an electron withdrawing group (such as a fluorine atom or a carboxyl group) may be used as a co-catalyst. The carbonyl compound may include, for example, hexafluoroacetone, trifluoroacetic acid, pentafluorophenyl ketone, and benzoic acid. The amount of the carbonyl compound is, for example, about 0.0001 to 3 mol, preferably about 0.0005 to 2.5 mol, and more preferably about 0.001 to 2 mol, relative to 1 mol of the reaction component (the substrate).

Furthermore, in order to accelerate the reaction, a radical-generating agent or a radical-reaction accelerator or promoter may be present in the system. Such a component may include, for example, a halogen (e.g., chlorine and bromine), a peracid (e.g., peracetic acid and m-chloroperbenzoic acid), a peroxide (e.g., hydrogen peroxide and a hydroperoxide such as t-butylhydroperoxide (TBHP)), nitric acid or nitrous acid or a salt thereof, nitrogen dioxide, and an aldehyde such as benzaldehyde (e.g., an aldehyde corresponding to the aromatic polycarboxylic acid as an object compound). The amount of the component is about 0.001 to 1 mol, preferably about 0.005 to 0.8 mol, and more preferably about 0.01 to 0.5 mol, relative to 1 mol of the imide compound.

[Substrate]

In the present invention, an aromatic compound having a plurality of alkyl groups is used as a substrate. Incidentally, throughout this description, the “alkyl group” of the substrate includes not only an alkyl group but also a “lower-order oxidized group” of the alkyl group, which is produced by oxidation of the alkyl group and has not yet formed a final carboxyl group or an equivalent thereof (e.g., an acid anhydride group).

In the present invention, the aromatic compound usually has an alkyl group corresponding to the aromatic carboxylic acid. Thus the aromatic compound may have 2 to 6 (particularly 3 to 6) alkyl groups as substituents on a benzene ring thereof or may have 4 to 8 (particularly 4 to 6) alkyl groups as substituents on a naphthalene ring. When the aromatic compound is a compound having a biphenyl skeleton (a biphenyl compound), the compound may have 4 to 10 (particularly 4 to 6) alkyl groups as substituents on the biphenyl skeleton. When the aromatic compound is a compound having a triphenyl skeleton (a terphenyl compound), the compound may have 6 to 15 (particularly 4 to 8) alkyl groups as substituents on the triphenyl skeleton. The aromatic compound usually has about 2 to 10 (preferably about 3 to 6, and more preferably about 3 to 5) of alkyl groups or lower-order oxidized groups thereof on the aromatic ring.

The aromatic ring may include an aromatic hydrocarbon ring [for example, a monocyclic or condensed polycyclic hydrocarbon ring corresponding to benzene, naphthalene, acenaphtylene, phenanthrene, anthracene, pyrene, and the like; a ring-assembly hydrocarbon ring, e.g., a hydrocarbon ring corresponding to biphenyl, terphenyl, binaphthyl, and the like; and a bisarene compound in which aromatic hydrocarbon rings are linked through a bivalent group such as an oxygen atom, a sulfur atom, a sulfide group, a carbonyl group, an alkylene group, or a cycloalkylene group, e.g., a bisarene corresponding to biphenyl ether, biphenyl sulfide, biphenyl sulfone, biphenyl ketone, a biphenylalkane, and the like]; and an aromatic heterocycle having about one to three hetero atoms comprising at least one selected independently from the group consisting of an oxygen atom, a sulfur atom, and a nitrogen atom [for example, a thiophene ring, a pyrrole ring, an imidazole ring, an oxazole ring, a thiazole ring, a pyridine ring, a pyridazine ring, a pyrimidine ring, a pyrazine ring, a quinoline ring, an indole ring, an indazole ring, a benzotriazole ring, a quinazoline ring, an acridine ring, and a chromone ring]. Each of these aromatic rings may have a substituent (for example, a carboxyl group, a halogen atom, a hydroxyl group, an alkoxy group, an acyloxy group, a substituted oxycarbonyl group, a substituted or non-substituted amino group, and a nitro group). Moreover, the aromatic ring may be condensed with a ring having an aromatic property of a ring having a non-aromatic property.

The alkyl group bonding to the aromatic ring may include, for example, a primary or secondary C₁₋₁₀alkyl group such as methyl, ethyl, propyl, isopropyl, butyl, isobutyl, s-butyl, pentyl, isopentyl, hexyl, isohexyl, heptyl, octyl, 2-ethylhexyl, or decyl group. The preferred alkyl group includes a C₁₋₄alkyl group, particularly a C₁₋₃alkyl group such as methyl group, ethyl group, or isopropyl group. The lower-order oxidized group of the alkyl group may include, for example, a hydroxyalkyl group (e.g. , a hydroxyC₁₋₃alkyl group such as hydroxymethyl or 1-hydroxyethyl group), a formyl group, a formylalkyl group (e.g. , a formylC₁₋₃alkyl group such as formylmethyl or 1-formylethyl group), and an alkyl group having an oxo group (e.g., a C₁₋₄acyl group such as acetyl, propionyl, or butyryl group). The alkyl group or the lower-order oxidized group thereof may have a substituent as long as the reaction is not inhibited.

The aromatic compound may include a compound having two alkyl groups [for example, a xylene (o- , m- , p-xylene), 1-ethyl-4-methylbenzene, 1-ethyl-3-methylbenzene, a xylenol (e.g., 2,3-, 2,4-, or 3,5-xylenol), thymol (6-isopropyl-m-cresol), methylbenzaldehyde, a dimethylbenzoic acid (e.g., 2,3-, 2,4-, or 3,5-dimethylbenzoic acid), 4,5-dimethylphthalic acid, 4,6-dimethylisophthalic acid, 2,5-dimethylterephthalic acid, a dimethylnaphthalene (e.g., 1,5-, or 2,5-dimethylnaphthalene), dimethylanthracene, 4,4′-dimethylbiphenyl, a dimethylpyridine (2,3-lutidine, 2,4-lutidine, 2,5-lutidine, 3,5-lutidine, 2,6-lutidine), 2-ethyl-4-methylpyridine, 3,5-dimethyl-4-pyrone, and an N-substituted or non-substituted 3,5-dimethyl-4-pyridone]; a compound having three alkyl groups [for example, 1,2,3-trimethylbenzene, 1,2,4-trimethylbenzene (pseudocumene), 1,3,5-trimethylbenzene (mesitylene), dimethylbenzyl alcohol, dimethylbenzaldehyde, 2,4,5-trimethylbenzoic acid, trimethylanthracene, and a trimethylpyridine (e.g., 2,3,4-, 2,3,5-, 2,3,6-, or 2,4,6-trimethylpyridine)]; a compound having four or more alkyl groups [for example, a compound having a plurality of alkyl substituents on one benzene ring or naphthalene ring thereof, e.g., 1,2,3,5-tetramethylbenzene, 1,2,3,4-tetramethylbenzene, 1,2,4,5-tetramethylbenzene (durene), pentamethylbenzene, 1,2,3,4,5,6-hexamethylbenzene, or a tetramethylnaphthalene (e.g., 1,2,5,6-, 2,3,6,7-, or 1,2,7,8-tetramethylnaphthalene); and a biphenyl compound having a plurality of alkyl groups on each of a plurality of benzene rings thereof, e.g., atetramethylbiphenyl (e.g., 3,3′,4,4′-, or 2,2′,3,3′-tetramethylbiphenyl), a tetramethylbiphenyl ether (e.g., bis(3,4-dimethylphenyl) ether, bis(2,3-dimethylphenyl) ether, and 2,3,3′,4′-tetramethyldiphenyl ether), a tetramethylbiphenyl sulfide (e.g., bis(3,4-dimethylphenyl) sulfide, and bis(2,3-dimethylphenyl) sulfide), a tetramethylbiphenyl sulfone (e.g., bis(3,4-dimethylphenyl) sulfone, and bis(2,3-dimethylphenyl) sulfone), a tetramethylbiphenyl ketone (e.g., bis(3,4-dimethylphenyl) ketone, bis(2,3-dimethylphenyl) ketone, and 2,3,3′,4′-tetramethyldiphenyl ketone), a tetramethylbiphenylalkane (e.g., a bis(3,4-dimethylphenyl)C₁₋₆alkane, a bis(2,3-dimethylphenyl)C₁₋₆alkane, and a 2,3,3′,4′-tetramethyldiphenylC₁₋₆alkane), a tetramethylbiphenylcycloalkane (e.g., a bis(3,4-dimethylphenyl)C₄₋₁₀ cycloalkane, and a bis(2,3-dimethylphenyl)C₄₋₁₀cycloalkane), 2,3,4,3′,4′-pentamethyl diphenyl ether, and 2,3,4,3′,4F-pentamethyl diphenyl ketone]. The aromatic compound may be used alone or in combination.

The preferred aromatic compound includes a compound having three or more alkyl groups. Moreover, the aromatic compound preferably has at least two alkyl groups on ortho position of an aromatic ring thereof. Incidentally, when the aromatic compound having a plurality of aromatic rings has a plurality of alkyl groups on each of the plurality of aromatic rings (for example, two benzene rings), these alkyl groups may be located in symmetry or asymmetry. Such an aromatic compound may include, for example, pseudocumene (1,2,4 - trimethylbenzene), durene, hexamethylbenzene, a polyalkylnaphthalene [for example, a dimethylnaphthalene (e.g., 1,2-dimethylnaphthalene and 2,3-dimethylnaphthalene), a trimethylnaphthalene (e.g., 1,2,4-trimethylnaphthalene), and a tetramethylnaphthalene (e.g., 1,2,3,4-tetramethylnaphthalene, 1,2,5,6-tetramethylnaphthalene, and 2,3,6,7-tetramethylnaphthalene) ], a polyalkylbisaryl [for example, a tetramethylbiphenyl (e.g., 2,3,4,5-tetramethylbiphenyl, 2,3,11,12-tetramethylbiphenyl, and 3,4,10,11-tetramethylbiphenyl); and a tetramethylbiphenyl ether, a tetramethylbiphenyl sulfone, a tetramethylbiphenyl ketone, a tetramethylbiphenylalkane and a tetramethylbiphenylcycloalkane, each corresponding to the tetramethylbiphenyl].

According to the process of the present invention, the aromatic polycarboxylic acid can be obtained in a high yield by oxidizing the plurality of alkyl groups of the aromatic ring efficiently. For example, trimellitic acid and/or trimellitic anhydride from pseudocumene; pyromellitic acid and/or pyromellitic anhydride from durene; and 3,3′,4,4′-benzophenone-tetracarboxylic acid from 3,3′,4,4′-tetramethylbenzophenone can be individually obtained in a high yield. In particular, according to the conventional process, an aromatic compound having an alkyl group as an intermediate product of an oxidation reaction remains in large quantity, and it is difficult to produce an aromatic polycarboxylic acid as an object compound efficiently. To take oxidation of durene as an example for explanation, a monocarboxylic acid, a dicarboxylic acid, a tricarboxylic acid, a tetracarboxylic acid, or an acid anhydride thereof is produced with oxidation of the substrate. The activity to the oxidation is as follows in decreasing order: substrate>monocarboxylic acid>dicarboxylic acid>tricarboxylic acid. On the other hand, the transition metal co-catalyst tends to form a salt with the following carboxylic acid easily in increasing order: monocarboxylic acid<dicarboxylic acid<tricarboxylic acid<tetracarboxylic acid, and in some cases, the salt is precipitated as an insoluble matter. The transition metal co-catalyst is consumed by forming a salt with the carboxylic acid, and the catalyst activity is remarkably decreased. Therefore, an aromatic compound having an alkyl group and a carboxyl group remains in a relatively large quantity, and it is difficult to improve the efficiency of the oxidation from a tricarboxylic acid (methyltrimellitic acid) to pyromellitic acid (in which all alkyl groups of the tricarboxylic acid are converted into carboxyl groups). In particular, it is difficult to allow the reaction to proceed at a latter stage of the reaction. In the present invention, the aromatic polycarboxylic acid can be produced efficiently even in such a reaction system. In these respects, it is preferable that in the present invention the metal co-catalyst be added (particularly, successively or continuously added, e.g., by dropwise) to the reaction system (particularly, the reaction system at least at the latter stage) in order to avoid the consumption of the transition metal co-catalyst or the deactivation of the catalyst (the imide compound). Moreover, along with the addition (or supplementation) of the metal co-catalyst, the catalyst (the imide compound) may be added (particularly, successively or continuously added, e.g., by dropwise) to the reaction system (particularly, the reaction system at least at the latter stage). Incidentally, the temperature zone in which the reaction is conducted to reach the oxidation degree of not less than 30% may be considered as an initial stage of the reaction, and the temperature zone in which the reaction is conducted to reach the oxidation degree of not less than 75% may be considered as a latter stage of the reaction. Therefore, the addition of the metal co-catalyst and/or the catalyst (the imide compound) may be conducted at either the initial stage of the reaction or the latter stage of the reaction. As described above, the addition of the metal co-catalyst and/or the catalyst at least at the latter stage of the reaction is advantageous.

Incidentally, the aromatic compound as the substrate can be introduced into the reaction system by feeding at a time at the initial stage, successive addition, continuous addition, and others.

[Oxygen]

As the oxygen, any of a molecular oxygen and a nascent oxygen may be used. The molecular oxygen is not particularly limited to a specific one, and may include a pure oxygen or an oxygen diluted with an inactive (or inert) gas (e.g., nitrogen, helium, argon, and carbon dioxide), an air, and a diluted air. Moreover, the oxygen may be generated in the system. The amount of the oxygen is usually not less than 0.5 mol (e.g., not less than 1 mol), preferably about 1 to 10000 mol, and more preferably about 5 to 1000 mol, relative to 1 mol of the substrate. The molar quantity of the oxygen is in excess of the molar quantity of the substrate in practical cases.

The oxygen can be introduced into the reaction system in various forms (or modes) such as a continuous feeding, a successive feeding, and a bulk feeding (or batch feeding). In the preferred process, the oxygen is fed to the reaction system continuously. Incidentally, the concentration of the off-gas oxygen from the reaction system is not particularly limited to a specific one, and is, for example, about 0 to 20% by volume (e.g., about 0.5 to 10% by volume), and usually about 1 to 9% by volume.

[Reaction Solvent]

The reaction may be carried out in the absence of a solvent, and is usually carried out in the presence of a solvent. As the solvent, there may be, for example, an aromatic hydrocarbon such as benzene; a halogenated hydrocarbon such as dichloromethane, chloroform, 1,2-dichloroethane, or dichlorobenzene; an alcohol such as t-butanol or t-amyl alcohol; a nitrile such as acetonitrile or benzonitrile; an organic acid such as formic acid, acetic acid, propionic acid, butyric acid, isobutyric acid, or hexanoic acid; an ester such as ethyl acetate; and an amide such as formamide, acetamide, dimethylformamide (DMF), or dimethylacetamide. These solvents may be used as a mixed solvent. Among these solvents, a protic organic solvent (e.g., an organic acid) and a nitrile, particularly acetic acid is preferred for reactivity and economic reasons. The amount of the reaction solvent is about 1.5 to 100 times, preferably about 3 to 50 times, and more preferably about 5 to 25 times as large as the amount of the substrate (the aromatic compound).

[Acid Anhydride ]

If necessary, an acid anhydride may be added to the reaction system. The acid anhydride may include, for example, an aliphatic monocarboxylic anhydride such as acetic anhydride, propionic anhydride, butyric anhydride, or isobutyric anhydride; an aromatic monocarboxylic anhydride such as benzoic anhydride; and the acid anhydride as described in the paragraph of the catalyst (e.g., an aliphatic polycarboxylic anhydride, an alicyclic polycarboxylic anhydride, and an aromatic polycarboxylic anhydride). Among these acid anhydrides, an aliphatic monocarboxylic anhydride, particularly an acetic anhydride, is preferred. The amount of the acid anhydride may be, for example, about 0.1 to 100 mol, preferably about 0.5 to 40 mol, and more preferably about 1 to 20 mol, relative to 1 mol of the substrate (the aromatic compound). A largely excessive amount of the acid anhydride may be used relative to the amount of the substrate.

[Production Process ]

According to the present invention, in a process comprising oxygen-oxidizing the substrate (the aromatic compound) under heating in the presence of a catalyst system comprising the catalyst and the metal co-catalyst, the oxygen-oxidation is conducted by heating in a plurality of temperature zones with feeding the oxygen continuously. Incidentally, in the reaction system, the catalyst and the oxygen may be fed continuously in the presence of the metal co-catalyst. Assuming that the oxidation degree of the aromatic compound having a plurality of alkyl groups (e.g., methyl groups) as a substrate is 0% and the oxidation degree of a compound in which all alkyl groups (e.g., methyl groups) of the aromatic compound are oxidized into carboxyl groups is 100%, the plurality of temperature zones contains at least two temperature zones comprising a first temperature zone (lower-temperature zone) for conducting a reaction to reach the oxidation degree of not less than 30% (e.g., 30 to 70%) and a second temperature zone (higher-temperature zone) for conducting a reaction to reach the oxidation degree of not less than 75%. The reaction in such a plurality of temperature zones can efficiently avoid the consumption of the metal co-catalyst and the lowering of the catalyst activity, and can obtain an aromatic polycarboxylic acid in a high yield. In contrast, when the reaction is conducted to reach the oxidation degree of not less than 75% in the initial stage of the reaction (oxidation), a salt of the metal co-catalyst with a polycarboxylic acid produced in the reaction system is rapidly formed, and the catalyst activity is remarkably deteriorated. Therefore, it is difficult to increase the yield of the aromatic polycarboxylic acid. Incidentally, the lower-temperature zone and the higher-temperature zone can be considered as the initial stage of the reaction and the latter stage of the reaction, respectively.

By way of an example in which the number of alkyl groups of the aromatic compound is n, the oxidation degree will be explained as follows. When the oxidation reaction product consists of a polycarboxylic acid having n carboxyl groups (yield: m₁%), a polycarboxylic acid having n−1 carboxyl groups (yield: m₂%), a polycarboxylic acid having n−2 carboxyl groups (yield: m₃%), . . . , and a carboxylic acid having n−(n−1) carboxyl group (yield: mₓ%) based on HPLC analysis, the oxidation degree can be calculated on the basis of the following equation:

Oxidation degree=m₁%+m₂%×(n−1/n)+m₃%×(n−2/n)+ . . . +mₓ%×(1/n)

When the aromatic compound is durene (the number of methyl groups: 4), the oxidation degree can be calculated on the basis of the following equation:

Oxidation degree=m₁%+m₂%×(¾)+m₃%×( 2/4)+m₄%×(¼)

Incidentally, since oxidation products (such as an aldehyde or an alcohol) other than the above-mentioned carboxylic acids are produced, an actual oxidation degree is higher than the calculation value obtained on the basis of the above-mentioned equation. However, the components are left out of consideration.

In the lower-temperature zone, it is sufficient that the reaction is conducted to reach the oxidation degree of not less than 30%. Usually, the reaction is conducted to reach the oxidation degree of about 35 to 75% (e.g., about 35 to 65%), preferably about 45 to 70%, and usually about 40 to 65% (e.g., about 45 to 60%). The oxidation degree in the lower-temperature zone may be about 50 to 75% (e.g., about 50 to 70%) and particularly about 50 to 65% (e.g., about 50 to 60%).

The reaction in the lower-temperature zone (the initial stage of the reaction) may be conducted in a single reaction temperature zone or in a plurality of reaction temperature zones obtained by raising the temperature stepwisely, or may be conducted by raising the temperature continuously. Depending on the species of the substrate (the aromatic compound), the reaction in the lower-temperature zone can usually be carried out at a reaction temperature of about 50 to 140° C. (e.g., about 60 to 135° C., preferably about 65 to 130° C., and more preferably about 70 to 120° C.), and the reaction temperature may be about 70 to 130° C. Moreover, the reaction in the lower-temperature zone may be carried out at about 60 to 120° C., preferably about 65 to 100° C., and more preferably at about 70 to 90° C.

In order to produce the aromatic polycarboxylic acid (for example, an aromatic polycarboxylic acid having an oxidation degree of 75 to 100% , particularly, an oxidation degree of 85 to 100%) with preventing the consumption of the metal co-catalyst and the deterioration of the catalyst activity, the lower-temperature zone (the initial stage of the reaction) preferably contains at least a first lower-temperature zone of not higher than 120° C. [for example, lower than 120° C. (e.g., lower than 100° C.)]. The reaction temperature in the first lower-temperature zone is, for example, about 60 to 120° C. (e.g., about 60 to 115° C.), preferably about 70 to 110° C. (e.g., about 75 to 90° C.), and usually about 60 to 110° C. The reaction temperature in the first lower-temperature zone is about 60 to 95° C. and preferably about 70 to 90° C. (e.g., about 75 to 90° C.), and may usually be about 60 to 90° C.

It is preferable that the lower-temperature zone contain a second lower-temperature zone (or an intermediate temperature zone) for conducting a reaction subsequent to the reaction in the first lower-temperature zone. The reaction temperature in the second lower-temperature zone (or the intermediate temperature zone) is usually higher than the reaction temperature in the first lower-temperature zone, and may be, for example, about 100 to 140° C. (e.g., about 105 to 135° C.) and preferably about 110 to 130° C. (e.g., about 115 to 125° C.). Incidentally, in the lower-temperature zone (the first lower-temperature zone and/or the second lower-temperature zone), the reaction temperature may be raised stepwisely or continuously.

The reaction in such a lower-temperature zone can effectively inhibit a salt formation of the polycarboxylic acid produced in the reaction system with the metal co-catalyst. Incidentally, when the reaction is conducted at a high temperature from the initial stage of the reaction, a rapid salt formation of the metal co-catalyst with the polycarboxylic acid produced in the reaction system remarkably deteriorates the catalyst activity. For example, when pyromellitic acid and the transition metal co-catalyst are heated and stirred at 110° C. in acetic acid, a metal salt is formed in about one minute. On the other hand, when pyromellitic acid and the transition metal co-catalyst are heated and stirred at 90° C. in acetic acid, it takes about 5 minutes before a metal salt is formed. Therefore, depending on the substrate (the aromatic compound), it is preferable that the reaction be conducted in a lower-temperature zone in the initial stage of the reaction and then in a higher-temperature zone.

In the higher-temperature zone, it is sufficient that the substrate is conducted to reach the oxidation degree of not less than 75%. Usually, the reaction is conducted to reach the oxidation degree of not less than 80% (about 80 to 100%, for example, about 85 to 99%). The reaction in the higher-temperature zone is usually conducted, at a temperature higher than the reaction temperature in the lower-temperature zone, subsequent to the reaction in the lower-temperature zone (e.g., the second lower-temperature zone). The reaction temperature in the higher-temperature zone is about 100 to 150° C., preferably about 110 to 150° C. (e.g., about 115 to 145° C.), and more preferably about 120 to 140° C. In the higher-temperature zone (the latter stage of the reaction), the reaction temperature may be raised stepwisely or continuously.

Incidentally, in addition to the lower-temperature zone (the first lower-temperature zone, the second lower-temperature zone) and the higher-temperature zone, the plurality of temperature zones may contain a further temperature zone between the above-mentioned temperature zones, and in the further temperature zone the reaction is conducted at an intermediate temperature between temperatures in the above-mentioned temperature zones. Moreover, the plurality of temperature zones may contain a further temperature zone higher than the higher-temperature zone, for conducting a reaction subsequent to the reaction in the higher-temperature zone. Moreover, the reaction temperature may be raised to a preset temperature in a preset time by a heating program. Furthermore, the width of the temperature rise is not particularly limited to a specific one as long as the temperature is in a predetermined temperature zone, and may be about 1 to 10° C. The number of steps (or stages) of the temperature rise is not particularly limited to a specific one, and may be about 2 to 10.

The reaction may be carried out under an atmospheric pressure (0.1 MPa), and is usually carried out under an applied pressure system. The reaction pressure may be, for example, about 0.3 to 20 MPa, preferably about 0.5 to 10 MPa, and more preferably about 0.6 to 5 MPa.

The reaction may be conducted by a conventional manner, for example, a reaction manner such as a batch operation, a semi-batch operation, or a continuous operation. After the reaction is completed, the reaction product may be separated and purified by a separation means (e.g., filtration, condensation, distillation, extraction, crystallization, recrystallization, adsorption, and column chromatography) or a combination means thereof.

According to the process of the present invention, since the aromatic compound having a plurality of alkyl groups is oxygen-oxidized in the presence of the catalyst system comprising the catalyst (the imide compound) and the transition metal co-catalyst under a predetermined condition, the aromatic polycarboxylic acid can be obtained highly selectively in a high yield. Therefore, the present invention is useful as a method for increasing the selective production of the aromatic polycarboxylic acid.

### INDUSTRIAL APPLICABILITY

The aromatic polycarboxylic acid or the acid anhydride thereof obtained by the present invention can be used in a variety of fields (e.g., the field of electronic industry materials), for example, a main raw material for a heat-resistant polymer (e.g., a polyimide-series polymer and a polyester-series polymer) and a heat-resistant plasticizer, and a hardening agent for a heat-resistant epoxy resin.

### EXAMPLES

Hereinafter, the following examples are intended to describe this invention in further detail and should by no means be interpreted as defining the scope of the invention.

Example 1

### Reaction Temperature 80° C.→120° C.→130° C.

Into an air-flow reactor, 40 g (0.30 mol) of durene, 320 g of acetic acid, 0.19 g (0.7 mmol) of cobalt acetate (bivalent), 0.55 g (2.2 mmol) of manganese acetate (bivalent), and 1.06 g (3.0 mmol) of zirconium sulfate were charged. The reactor pressure was raised to 0.8 MPa by nitrogen, and the mixture was heated to 80° C.

A slurry having 14.8 g (60 mmol) of N,N′ -dihydroxypyromellitimide added in 300 g of acetic acid and a mixed gas of air and nitrogen were fed to the reactor, and at this time the reaction was initiated. The slurry was fed to the reactor over 5 hours by a slurry pump, and the gas was fed to the reactor to adjust the oxygen concentration contained in off-gas to 2 to 8%. After the reaction was initiated, the reaction temperature was raised to 120° C. over 0.5 hours and then continued being maintained for 1.5 hours. At this point in time, a sample for HPLC analysis was taken. Thereafter, the reaction was continued for 3 hours at 130° C. During the reaction, the feed rate of the gas and that of the catalyst were adjusted, if necessary, to control the reaction.

After the catalyst addition was completed (5 hours after the reaction was initiated), the reaction mixture was aged at 130° C. for one hour with maintaining the oxygen concentration contained in off-gas at 8%. Thereafter, the supply of the gas was stopped, and the reactor was cooled and the pressure was released.

The results of the HPLC analysis at 2 hours after the reaction was initiated revealed that pyromellitic acid (yield 5% (3.8 g)), methyltrimellitic acid (yield 50% (33.4 g)), and dimethylterephthalic acid (yield 25% (14.5 g)) were produced. The oxidation degree at this point in time was as follows: 5%+50%×¾+25%×½=55%. Moreover, the HPLC analysis of the reaction mixture after the pressure was released revealed that pyromellitic acid (yield 86% (65.2 g)) and methyltrimellitic acid (yield 3% (2.0 g)) were produced.

### Reference Example 1

### Reaction Temperature 130° C.→160° C.

Into an air-flow reactor, 40 g (0.30 mol) of durene, 320 g of acetic acid, 0.19 g (0.7 mmol) of cobalt acetate (bivalent), 0.55 g (2.2 mmol) of manganese acetate (bivalent), and 1.06 g (3.0 mmol) of zirconium sulfate were charged. The reactor pressure was raised to 0.8 MPa by nitrogen, and the mixture was heated to 130° C.

A slurry having 14.8 g (60 mmol) of N,N′-dihydroxypyromellitimide added in 300 g of acetic acid and a mixed gas of air and nitrogen were fed to the reactor, and at this time the reaction was initiated. The slurry was fed to the reactor over 5 hours by a slurry pump, and the gas was fed to the reactor to adjust the oxygen concentration contained in off-gas to 2 to 8%. After the reaction was initiated, the reaction temperature was raised to 160° C. over 0.5 hours and then continued being maintained for 4.5 hours. During the reaction, the feed rate of the gas and that of the catalyst were adjusted, if necessary, to control the reaction.

After the catalyst addition was completed (5 hours after the reaction was initiated), the reaction mixture was aged at 160° C. for one hour with maintaining the oxygen concentration contained in off-gas at 8%. Thereafter, the supply of the gas was stopped, and the reactor was cooled and the pressure was released.

The HPLC analysis of the reaction mixture after the pressure was released revealed that pyromellitic acid (yield 44% (33.4 g)) and methyltrimellitic acid (yield 35% (23.4 g)) were produced.

### Example 2

### Reaction Temperature 80° C.→120° C.→130° C., Pressure 2 MPa

Into an air-flow reactor, 40 g (0.30 mol) of durene, 320 g of acetic acid, 0.19 g (0.7 mmol) of cobalt acetate (bivalent), 0.55 g (2.2 mmol) of manganese acetate (bivalent), and 1.06 g (3.0 mmol) of zirconium sulfate were charged. The reactor pressure was raised to 2 MPa by nitrogen, and the mixture was heated to 80° C.

A slurry having 14.8 g (60 mmol) of N,N′-dihydroxypyromellitimide added in 300 g of acetic acid and a mixed gas of air and nitrogen were fed to the reactor, and at this time the reaction was initiated. The slurry was fed to the reactor over 5 hours by a slurry pump, and the gas was fed to the reactor to adjust the oxygen concentration contained in off-gas to 2 to 8%. After the reaction was initiated, the reaction temperature was raised to 120° C. over 0.5 hours and then continued being maintained for 1.5 hours. At this point in time, a sample for HPLC analysis was taken. Thereafter, the reaction was continued for 3 hours at 130° C. During the reaction, the feed rate of the gas and that of the catalyst were adjusted, if necessary, to control the reaction.

After the catalyst addition was completed (5 hours after the reaction was initiated), the reaction mixture was aged at 130° C. for one hour with maintaining the oxygen concentration contained in off-gas at 8%. Thereafter, the supply of the gas was stopped, and the reactor was cooled and the pressure was released.

The results of the HPLC analysis at 2 hours after the reaction was initiated revealed that pyromellitic acid. (yield 8% (6.1 g)), methyltrimellitic acid (yield 50% (33.4 g)), and dimethylterephthalic acid (yield 23% (13.3 g)) were produced. The oxidation degree at this point in time was as follows: 8%+37.5%+11.5%=57%. Moreover, the HPLC analysis of the reaction mixture after the pressure was released revealed that pyromellitic acid (yield 91% (68.9 g)) and methyltrimellitic acid (yield 2% (1.3 g)) were produced.

### Comparative Example 1

### Reaction Temperature 60° C.→70° C.→90° C.

Into an air-flow reactor, 40 g (0.30 mol) of durene, 320 g of acetic acid, 0.19 g (0.7 mmol) of cobalt acetate (bivalent), 0.55 g (2.2 mmol) of manganese acetate (bivalent), and 1.06 g (3.0 mmol) of zirconium sulfate were charged. The reactor pressure was raised to 0.8 MPa by nitrogen, and the mixture was heated to 60° C.

A slurry having 14.8 g (60 mol) of N,N′-dihydroxypyromellitimide added in 300 g of acetic acid and a mixed gas of air and nitrogen were fed to the reactor, and at this time the reaction was initiated. The slurry was fed to the reactor over 5 hours by a slurry pump, and the gas was fed to the reactor to adjust the oxygen concentration contained in off-gas to 2 to 8%. After the reaction was initiated, the reaction temperature was raised to 70° C. over 0.5 hours and then continued being maintained for 1.5 hours. At this point in time, a sample for HPLC analysis was taken. Thereafter, the reaction was continued for 3 hours at 90° C. During the reaction, the feed rate of the gas and that of the catalyst were adjusted, if necessary, to control the reaction.

After the catalyst addition was completed (5 hours after the reaction was initiated), the reaction mixture was aged at 90° C. for one hour with maintaining the oxygen concentration contained in off-gas at 8%. Thereafter, the supply of the gas was stopped, and the reactor was cooled and the pressure was released.

The results of the HPLC analysis at 2 hours after the reaction was initiated revealed that the oxidation degree at this point in time was 20% (the yield of trimethylbenzoic acid: 50%, and the yield of dimethylterephthalic acid: 15%). Moreover, the HPLC analysis of the reaction mixture after the pressure was released revealed that pyromellitic acid (yield 3% (2.3 g)) and methyltrimellitic acid (yield 26% (17.4 g)) were produced.

### Example 3

### Zirconium Oxoacetate

Into an air-flow reactor, 40 g (0.30 mol) of durene, 320 g of acetic acid, 0.19 g (0.7 mmol) of cobalt acetate (bivalent), 0.55 g (2.2 mmol) of manganese acetate (bivalent), and 0.76 g (3.0 mmol) of zirconium oxoacetate were charged. The reactor pressure was raised to 0.8 MPa by nitrogen, and the mixture was heated to 80° C.

A slurry having 14.8 g (60 mmol) of N,N′-dihydroxypyromellitimide added in 300 g of acetic acid and a mixed gas of air and nitrogen were fed to the reactor, and at this time the reaction was initiated. The slurry was fed to the reactor over 5 hours by a slurry pump, and the gas was fed to the reactor to adjust the oxygen concentration contained in off-gas to 2 to 8%. After the reaction was initiated, the reaction temperature was raised to 120° C. over 0.5 hours and then continued being maintained for 1.5 hours. At this point in time, a sample for HPLC analysis was taken. Thereafter, the reaction was continued for 3 hours at 130° C. During the reaction, the feed rate of the gas and that of the catalyst were adjusted, if necessary, to control the reaction.

After the catalyst addition was completed (5 hours after the reaction was initiated), the reaction mixture was aged at 130° C. for one hour with maintaining the oxygen concentration contained in off-gas at 8%. Thereafter, the supply of the gas was stopped, and the reactor was cooled and the pressure was released.

The results of the HPLC analysis at 2 hours after the reaction was initiated revealed that pyromellitic acid (yield 7% (5.3 g)), methyltrimellitic acid (yield 51% (34.1 g)), and dimethylterephthalic acid (yield 23% (13.3 g)) were produced. The oxidation degree at this point in time was as follows: 7%+38.25%+11.5%=56.8%. Moreover, the HPLC analysis of the reaction mixture after the pressure was released revealed that pyromellitic acid (yield 87% (66.0 g)) and methyltrimellitic acid (yield 3% (2.0 g)) were produced.

### Example 4

### Pseudocumene

Into an air-flow reactor, 36 g (0.30 mol) of pseudocumene, 320 g of acetic acid, 0.19 g (0.7 mmol) of cobalt acetate (bivalent), 0.55 g (2.2 mmol) of manganese acetate (bivalent), and 0.76 g (3.0 mmol) of zirconium oxoacetate were charged. The reactor pressure was raised to 0.8 MPa by nitrogen, and the mixture was heated to 80° C.

A slurry having 14.8 g (60 mmol) of N,N′-dihydroxypyromellitimide added in 300 g of acetic acid and a mixed gas of air and nitrogen were fed to the reactor, and at this time the reaction was initiated. The slurry was fed to the reactor over 5 hours by a slurry pump, and the gas was fed to the reactor to adjust the oxygen concentration contained in off-gas to 2 to 8%. After the reaction was initiated, the reaction temperature was raised to 120° C. over 0.5 hours and then continued being maintained for 1.5 hours. At this point in time, a sample for HPLC analysis was taken. Thereafter, the reaction was continued for 3 hours at 130° C. During the reaction, the feed rate of the gas and that of the catalyst were adjusted, if necessary, to control the reaction.

After the catalyst addition was completed (5 hours after the reaction was initiated), the reaction mixture was aged at 130° C. for one hour with maintaining the oxygen concentration contained in off-gas at 8%. Thereafter, the supply of the gas was stopped, and the reactor was cooled and the pressure was released.

The results of the HPLC analysis at 2 hours after the reaction was initiated revealed that trimellitic acid (yield 42% (26.5 g)) and methylterephthalic acid (yield 40% (21.6 g)) were produced. The oxidation degree at this point in time was as follows: 42%+26.7%=68.7%. Moreover, the HPLC analysis of the reaction mixture after the pressure was released revealed that trimellitic acid (yield 91% (57.3 g)) and methylterephthalic acid (yield 2% (1.1 g)) were produced.

### Example 5

### 3,3′,4,4′-Tetramethylbenzophenone

Into an air-flow reactor, 71.5 g (0.30 mol) of 3,3′,4,4′-tetramethylbenzophenone, 320 g of acetic acid, 0.19 g (0.7 mmol) of cobalt acetate (bivalent), 0.55 g (2.2 mmol) of manganese acetate (bivalent), and 1.06 g (3.0 mmol) of zirconium sulfate were charged. The reactor pressure was raised to 0.8 MPa by nitrogen, and the mixture was heated to 80° C.

A catalyst mixture having 13.8 g (120 mmol) of N-hydroxysuccinimide added in 300 g of acetic acid and a mixed gas of air and nitrogen were fed to the reactor, and at this time the reaction was initiated. The catalyst mixture was fed to the reactor over 5 hours by a slurry pump, and the gas was fed to the reactor to adjust the oxygen concentration contained in off-gas to 2 to 8%. After the reaction was initiated, the reaction temperature was raised to 120° C. over 0.5 hours and then continued being maintained for 0.5 hours. At this point in time, a sample for HPLC analysis was taken. Thereafter, the reaction was continued for 4 hours at 130° C. During the reaction, the feed rate of the gas and that of the catalyst were adjusted, if necessary, to control the reaction.

After the catalyst addition was completed (5 hours after the reaction was initiated), the reaction mixture was aged at 130° C. for one hour with maintaining the oxygen concentration contained in off-gas at 8%. Thereafter, the supply of the gas was stopped, and the reactor was cooled and the pressure was released.

The results of the HPLC analysis at one hour after the reaction was initiated revealed that 3 -methylbenzophenone-3,4,4′-tricarboxylic acid (yield 10% (9.8 g)), 3,3′-dimethylbenzophenone -4,4′-dicarboxylic acid (yield 45% (40.2 g)), and 3,3′,4′-trimethylbenzophenone-4-carboxylic acid (yield 30% (24.1 g)) were produced. The oxidation degree at this point in time was as follows: 17.5%+22.5%+7.5%=37.5%. Moreover, the HPLC analysis of the reaction mixture after the pressure was released revealed that benzophenone-3,3′,4,4′-tetracarboxylic acid (yield 80% (86 g)) and 3′-methylbenzophenone-3,4,4′-tricarboxylic acid (yield 3% (3.0 g)) were produced.

### Comparative Example 2

### Reaction Temperature 120° C. Constant

Into an air-flow reactor, 40 g (0.30 mol) of durene, 320 g of acetic acid, 0.19 g (0.7 mmol) of cobalt acetate (bivalent), 0.55 g (2.2 mmol) of manganese acetate (bivalent), and 1.06 g (3.0 mmol) of zirconium sulfate were charged. The reactor pressure was raised to 0.8 MPa by nitrogen, and the mixture was heated to 120° C.

A slurry having 14.8 g (60 mmol) of N,N′ -dihydroxypyromellitimide added in 300 g of acetic acid and a mixed gas of air and nitrogen were fed to the reactor, and at this time the reaction was initiated. The slurry was fed to the reactor over 5 hours by a slurry pump, and the gas was fed to the reactor to adjust the oxygen concentration contained in off-gas to 2 to 8%. During the reaction, the feed rate of the gas and that of the catalyst were adjusted, if necessary, to control the reaction.

After the catalyst addition was completed (5 hours after the reaction was initiated), the reaction mixture was aged at 120° C. for one hour with maintaining the oxygen concentration contained in off-gas at 8%. Thereafter, the supply of the gas was stopped, and the reactor was cooled and the pressure was released.

The HPLC analysis of the reaction mixture after the pressure was released revealed that pyromellitic acid (yield 66% (50.3 g)) and methyltrimellitic acid (yield 24% (16.1 g)) were produced.

### Comparative Example 3

### Reaction Temperature 130° C. Constant

Into an air-flow reactor, 40 g (0.30 mol) of durene, 320 g of acetic acid, 0.19 g (0.7 mmol) of cobalt acetate (bivalent), 0.55 g (2.2 mmol) of manganese acetate (bivalent), and 1.06 g (3.0 mmol) of zirconium sulfate were charged. The reactor pressure was raised to 0.8 MPa by nitrogen, and the mixture was heated to 130° C.

A slurry having 14.8 g (60 mmol) of N,N′-dihydroxypyromellitimide added in 300 g of acetic acid and a mixed gas of air and nitrogen were fed to the reactor, and at this time the reaction was initiated. The slurry was fed to the reactor over 5 hours by a slurry pump, and the gas was fed to the reactor to adjust the oxygen concentration contained in off-gas to 2 to 8%. During the reaction, the feed rate of the gas and that of the catalyst were adjusted, if necessary, to control the reaction.

After the catalyst addition was completed (5 hours after the reaction was initiated), the reaction mixture was aged at 130° C. for one hour with maintaining the oxygen concentration contained in off-gas at 8%. Thereafter, the supply of the gas was stopped, and the reactor was cooled and the pressure was released.

The HPLC analysis of the reaction mixture after the pressure was released revealed that pyromellitic acid (yield 52% (39.6 g)) and methyltrimellitic acid (yield 26% (17.4 g)) were produced.

### Comparative Example 4

### Catalyst Bulk Feeding

Into an air-flow reactor, 40 g (0.30 mol) of durene, 14.8 g (60 mmol) of N,N′-dihydroxypyromellitimide, 320 g of acetic acid, 0.19 g (0.7 mmol) of cobalt acetate (bivalent), 0.55 g (2.2 mmol) of manganese acetate (bivalent), and 1.06 g (3.0 mmol) of zirconium sulfate were charged. The reactor pressure was raised to 0.8 MPa by nitrogen, and the mixture was heated to 80° C. Acetic acid (300 g) and a mixed gas of air and nitrogen were fed. to the reactor, and at this time the reaction was initiated. The acetic acid was fed to the reactor over 5 hours, and the gas was fed to the reactor to adjust the oxygen concentration contained in off-gas to 2 to 8%. After the reaction was initiated, the reaction temperature was raised to 120° C. over 0.5 hours and then continued being maintained for 1.5 hours. At this point in time, a sample for HPLC analysis was taken. Thereafter, the reaction was continued for 3 hours at 130° C. During the reaction, the feed rate of the gas and that of the catalyst were adjusted, if necessary, to control the reaction.

After the acetic acid addition was completed (5 hours after the reaction was initiated), the reaction mixture was aged at 130° C. for one hour with maintaining the oxygen concentration contained in off-gas at 8%. Thereafter, the supply of the gas was stopped, and the reactor was cooled and the pressure was released.

The results of the HPLC analysis at 2 hours after the reaction was initiated revealed that pyromellitic acid (yield 10%) and methyltrimellitic acid (yield 48%) were produced. The oxidation degree at this point in time was as follows: 10%+48%×¾+23%×½=57.5%. Moreover, the HPLC analysis of the reaction mixture after the pressure was released revealed that pyromellitic acid (yield 12%) and methyltrimellitic acid (yield 48%) were produced.

1. A process for producing an aromatic polycarboxylic acid, which comprises oxygen-oxidizing an aromatic compound having a plurality of alkyl groups in the presence of a transition metal co-catalyst under heating in a plurality of temperature zones with feeding a catalyst and oxygen continuously,
the catalyst comprising a nitrogen atom-containing cyclic compound containing, as a constituent element of the cyclic ring, a skeleton represented by the following formula (1): 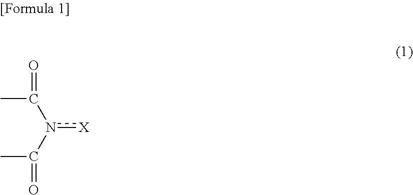
wherein X represents an oxygen atom or an −OR group (wherein R represents a hydrogen atom or a protecting group for a hydroxyl group), and a double line consisting of a solid line and a broken line and connecting “N” and “X” represents a single bond or a double bond,
wherein, assuming that the oxidation degree of the aromatic compound having a plurality of alkyl groups as a substrate is 0% and the oxidation degree of a compound in which all alkyl groups of the aromatic compound are oxidized into carboxyl groups is 100%, the plurality of temperature zones contains at least two temperature zones comprising
a first temperature zone for conducting a reaction to reach the oxidation degree of not less than 30% and
a second temperature zone for conducting a reaction to reach the oxidation degree of not less than 75%.

2. A production process according to claim 1, wherein the plurality of temperature zones contains
a lower-temperature zone for conducting a reaction at a reaction temperature within 50 to 140° C. to reach the oxidation degree of 35 to 65% and
a higher-temperature zone for conducting a reaction at a reaction temperature which is higher than the reaction temperature of the lower-temperature zone and is within 100 to 150° C. to reach the oxidation degree of not less than 80%.

3. A production process according to claim 2, wherein the lower-temperature zone contains at least a first lower-temperature zone for conducting a reaction at a reaction temperature of not higher than 120° C.

4. A production process according to claim 1, wherein the plurality of temperature zones contains
a first lower-temperature zone for conducting a first reaction at a reaction temperature within 60 to 120° C.,
a second lower-temperature zone (an intermediate temperature zone) for conducting a second reaction at a reaction temperature within 100 to 140° C. subsequent to the first reaction, and
a higher-temperature zone for conducting a third reaction at a reaction temperature within 110 to 150° C. subsequent to the second reaction.

5. A production process according to claim 1, wherein the transition metal co-catalyst comprises a metal component of the Group 9 of the Periodic Table of Elements, a metal component of the Group 7 of the Periodic Table of Elements, and a metal component of the Group 4 of the Periodic Table of Elements.

6. A production process according to claim 1, wherein the transition metal co-catalyst comprises a cobalt compound, a manganese compound, and a zirconium compound.

7. A production process according to claim 1, wherein the transition metal co-catalyst comprises a metal component of the Group 9 of the Periodic Table of Elements, a metal component of the Group 7 of the Periodic Table of Elements, and a metal component of the Group 4 of the Periodic Table of Elements, the ratio of the metal component of the Group 7 is 2 to 4 mol relative to 1 mol of the metal component of the Group 9 in terms of metal elements, and the ratio of the metal component of the Group 4 is 0.5 to 2 mol relative to 1 mol of the total amount of the metal component of the Group 9 and the metal component of the Group 7 in terms of metal elements.

8. A production process according to claim 2, wherein the reaction is conducted by adding the transition metal co-catalyst to the reaction system at least at the higher-temperature zone.

9. A production process according to claim 1, wherein the aromatic compound has 2 to 10 alkyl groups on an aromatic ring thereof.

10. A production process according to claim 1, wherein the catalyst has the same number of free carboxyl groups as the alkyl groups of the aromatic compound in the form of a free polycarboxylic acid corresponding to the catalyst.

11. A production process according to claim 1, wherein the catalyst corresponds to a tetracarboxylic anhydride and is an N-hydroxy cyclic imino compound in which a hydroxyl group may be protected.

12. A production process according to claim 1, wherein the aromatic polycarboxylic acid is pyromellitic acid.

13. A production process according to claim 1, wherein the oxidation reaction is conducted in a pressurized system.

14. A production process according to claim 1, which comprises
oxygen-oxidizing the aromatic compound having methyl groups on ortho position of an aromatic ring thereof in the presence of the transition metal co-catalyst under heating in a pressurized system with feeding a catalyst recited in claim 1 and oxygen continuously to produce the aromatic polycarboxylic acid having carboxyl groups on ortho position of an aromatic ring thereof,
wherein the transition metal co-catalyst comprises cobalt, manganese, and zirconium, and the number of moles of the zirconium is larger than the total molar quantity of the cobalt and the manganese, and
assuming that the oxidation degree of the aromatic compound having methyl groups as the substrate is 0% and the oxidation degree of a compound in which all methyl groups of the aromatic compound are oxidized into carboxyl groups is 100%, the oxidation reaction is conducted in the following temperature zones:
a first lower-temperature zone for conducting a reaction at a reaction temperature within 70 to 90° C.,
a second lower-temperature zone for conducting a reaction at a reaction temperature within 110 to 130° C. to reach the oxidation degree of 35 to 60%, and then
a higher-temperature zone for conducting a reaction at a reaction temperature within 120 to 140° C.

15. A method for increasing selective production of an aromatic polycarboxylic acid, which comprises oxygen-oxidizing an aromatic compound having a plurality of alkyl groups in the presence of a transition metal co-catalyst under heating in a plurality of temperature zones with feeding a catalyst recited in claim 1 and oxygen continuously,
wherein, assuming that the oxidation degree of the aromatic compound having a plurality of alkyl groups as a substrate is 0% and the oxidation degree of a compound in which all alkyl groups of the aromatic compound are oxidized into carboxyl groups is 100%, the plurality of temperature zones contains at least two temperature zones comprising
a first temperature zone for conducting a reaction to reach the oxidation degree of not less than 30% and
a second temperature zone for conducting a reaction to reach the oxidation degree of not less than 75%.
